(19) United States
(12) Patent Application Publication
(10) Pub. No.: US 2014/0322254 A1
(43) Pub. Date: Oct. 30, 2014

(54) POLYPEPTIDE ADJUVANT

(71) Applicant: University of Sheffield, Sheffield (GB)

(72) Inventors: Jon Sayers, Chesterfield (GB); Peter Arymiuk, Sheffield (GB); Andrew Heath, Sheffield (GB)

(73) Assignee: University of Sheffield, Sheffield (GB)

(21) Appl. No.: 14/360,680

(22) PCT Filed: Nov. 30, 2012
(86) PCT No.: PCT/GB2012/052976

§ 371 Date: May 27, 2014

(30) Foreign Application Priority Data
Nov. 30, 2011 (GB) 1120634.9

(51) Int. Cl. A61K 39/39; C07K 14/435; C07K 14/005; C07K 14/22; C07K 14/195

(52) U.S. Cl. CPC A61K 39/39; C07K 14/22; C07K 14/195; C07K 14/005; C07K 14/435; A61K 2039/55516
USPC 424/185.1; 530/350; 424/192.1

(57) ABSTRACT

The disclosure relates to an adjuvant polypeptide effective at enhancing the immune response to an antigen crosslinked to the adjuvant polypeptide.

The disclosure relates to an adjuvant polypeptide effective at enhancing the immune response to an antigen crosslinked to the adjuvant polypeptide or produced as a polypeptide fusion of antigen-polypeptide adjuvant or polypeptide-antigen fusion protein; vaccine compositions comprising said adjuvant and methods of vaccination that use said composition.

Adjuvants (immune potentiators or immunomodulators) have been used for decades to improve the immune response to vaccine antigens. The incorporation of adjuvants into vaccine formulations is aimed at enhancing, accelerating and prolonging the specific immune response to vaccine antigens. Advantages of adjuvants include the enhancement of the immunogenicity of weaker antigens, the reduction of the antigen amount needed for a successful immunisation, the reduction of the frequency of booster immunisations needed and an improved immune response in elderly and immunocompromised vaccinees.

Adjuvants can also be employed to optimise a desired immune response, e.g. with respect to immunoglobulin classes and induction of cytotoxic or helper T lymphocyte responses. In addition, certain adjuvants can be used to promote antibody responses at mucosal surfaces. Aluminium hydroxide and aluminium or calcium phosphate has been used routinely in human vaccines. More recently, antigens incorporated into IRIV's (immunostimulating reconstituted influenza virosomes) and vaccines containing the emulsion-based adjuvant MF59 have been licensed in certain territories. Adjuvants can be classified according to their source, mechanism of action and physical or chemical properties. The most commonly described adjuvant classes are gel-type, microbial, oil-emulsion and emulsifier-based, particulate, synthetic and cytokines.

More than one adjuvant may be present in the final vaccine product. They may be combined together with a single antigen or all antigens present in the vaccine, or each adjuvant may be combined with one particular antigen. The origin and nature of the adjuvants currently being used or developed is highly diverse. For example, aluminium based adjuvants consist of simple inorganic compounds, PLG is a polymeric carbohydrate, virosomes can be derived from disparate viral particles, MDP is derived from bacterial cell walls; saponins are of plant origin, squalene is derived from shark liver and recombinant endogenous immunomodulators are derived from recombinant bacterial, yeast or mammalian cells. There are several adjuvants licensed for veterinary vaccines, such as mineral oil emulsions that are too reactive for human use. Similarly, complete Freund's adjuvant, although being one of the most powerful adjuvants known, is not suitable for human use. There is a continual desire to identify adjuvants with reduced toxicity but with enhanced efficacy in promoting immune responses to antigens that are typically difficult to raise immune responses to.

Vaccines protect against a wide variety of infectious diseases. Many vaccines are produced by inactivated or attenuated pathogens which are injected into a subject. The immunised subject responds by producing both a humoral (e.g. antibody) and cellular (e.g. cytolytic T cells) responses. For example, some influenza vaccines are made by inactivating the virus by chemical treatment with formaldehyde. For many pathogens chemical or heat inactivation, while it may give rise to vaccine immunogens that confer protective immunity, also gives rise to side effects such as fever and injection site reactions. In the case of bacteria, inactivated organisms tend to be so toxic that side effects have limited the application of such crude vaccine immunogens (e.g. the cellular pertussis vaccine) and therefore vaccine development has lagged behind drug-development. Moreover, effective vaccine development using whole cell inactivated organisms suffers from problems of epitope masking, immunodominance, low antigen concentration and antigen redundancy. This is unfortunate as current antibiotic treatments are now prejudiced by the emergence of drug-resistant bacteria.

Many modern vaccines are therefore made from protective antigens of the pathogen, isolated by molecular cloning and purified from the materials that give rise to side-effects. These vaccines are known as ‘subunit vaccines’. The development of subunit vaccines has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens. However, although subunit vaccines tend to avoid the side effects of killed or attenuated pathogen vaccines, their ‘pure’ status means that subunit vaccines do not always have adequate immunogenicity to confer protection.

This disclosure relates to the identification of a family of bacterial polypeptides that have adjuvant activity when crosslinked or associated with an antigen to which an immune response is desired. The family is referred to as “α-protein”. The α-protein is a low complexity polypeptide so called because of the predicted alpha helical nature of its sequence. Relatively little is known about bacterial α-protein which is exposed to the host along with the extensively studied co-secreted IgA1 protease. The α-protein has been shown to enter mammalian cells where it passes through the cell membrane and cytoplasm. It eventually translocates into the nucleus but despite these intriguing observations little more has emerged regarding the function of α-protein. The protease is initially synthesized as a large pre-protein consisting of a leader peptide, large protease domain, a variable length α-protein and a C-terminal beta-barrel domain known as the β-core. After translocation into the periplasm, the β domain inserts into the outer membrane and facilitates auto-secretion of the protease and the α-protein. Once translocated through the outer membrane to the extracellular environment the protease domain cleaves itself from the membrane-embedded β-core at proline-rich recognition sites. Processing of further cleavage sites liberates free α-protein into the extracellular milieu where it is readily detectable in the secretions of those infected with the organism. The biological role of α-protein, other than acting as a physical linker between the mature protease and the β-core, is unknown.

We have found that physical association of α-protein with an antigen to which an immune response is desired results in an enhanced immune response to the antigen.

### STATEMENTS OF INVENTION

According to an aspect of the invention there is provided a vaccine composition comprising an adjuvant polypeptide wherein said adjuvant polypeptide comprises a bacterial α-protein cross-linked or associated with an antigenic molecule to which an immune response is desired.

In a preferred embodiment of the invention said α-protein comprises a polypeptide that includes at least one amino acid motif comprising amino acid residues EAERXAAELAXX(K/Q) wherein X is any hydrophilic amino acid residue.

In a preferred embodiment of the invention said α-protein includes at least 1, 2, 3, 4, 5, 6, 7, 8, 9, 10, 11, 12, 13, 14, 15, 16, 17, 18, 19 or 200 copies of the amino acid motif.

In a preferred embodiment of the invention said antigenic molecule is a polypeptide antigen.

In an alternative preferred embodiment of the invention said antigenic molecule is a polysaccharide antigen.

In a preferred embodiment of the invention said antigenic molecule is a lipopolysaccharide antigen.

In an alternative preferred embodiment of the invention α-protein and antigenic molecule are associated in a liposomal preparation.

In a preferred embodiment of the invention α-protein and antigen polypeptides are in frame translational fusion proteins.

In a preferred embodiment of the invention said antigen polypeptide is isolated from a bacterial pathogen.

In an alternative preferred embodiment of the invention said bacterial pathogen is a Gram negative bacterial pathogen.

In a preferred method of the invention said Gram negative bacteria is selected from the genus group consisting of: Neisseria, Moraxella, Escherichia, Salmonella, Shigella, Pseudomonas, Helicobacter, Legionella, Haemophilus, Klebsiella, Enterobacter, Cronobacter, Staphylococcus and Serratia.

Other species include Pseudomonas aeruginosa and other Pseudornonas species, Stenotrophomonas maltophila, Burkholdenia cepacia and other Burkholderia species, Aicatigenes xylosoxidans, species of Acinetobacter, Enterobacteriaceae, Haemophilus, Moraxella, Bacteroids, Fransicella, Shigelia, Proteus, Vibrio, Salmonella, Bordetella, Helicabactor, Legionella, Citrobactor, Campylobacter, Yersinia and Neisseria in another embodiment of the invention gram-negative bacteria include Enterobacteriaceae which is selected from the group consisting of organisms such as Serratia, Proteus, Klebsiella, Enterobacter, Citrobacter, Cronobacter, Salmonella, Providencia, Morganella, Cedecea and Escherichia coli.

In a preferred embodiment of the invention said human bacterial pathogen is Neisseria meningitidis.

In a preferred embodiment of the invention said human bacterial pathogen is Neisseria gonorrhoeae.

In a preferred embodiment of the invention said human bacterial pathogen is a Streptococcus species for example Streptococcus pneumonia.

In our co-pending application GB1102090.6 [unpublished] we disclose a class of protective antigen that advantageously induces the production of opsonins that target human bacterial pathogens. The Gly1 antigen is a secreted protein and shown to be essential to the growth of, for example, N. meningitidis when grown in defined media with haem or haemoglobin as the iron source. Gly1 is involved in iron metabolism and provides an essential function since the phenotype of deletion mutants in Gly1 is failure to grow under these conditions. The Gly1 protein is an example of a class of protein found in many pathogen bacterial species involved in haem sequestration and is likely involved in maintaining bacterial growth and the establishment of infection.

In a preferred embodiment of the invention said polypeptide is selected from the group consisting of:
i) an amino acid sequence selected from the group consisting of: SEQ ID NO: 1-26;
ii) an amino acid sequence as defined in i) above and which is modified by addition, deletion or substitution of one or more amino acid residues and which retains or has enhanced haem binding activity and/or reduced haemolytic activity.

A modified polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 35% identity, more preferably at least 40% identity, even more preferably at least 45% identity, still more preferably at least 50%, 60%, 70%, 80%, 90% identity, and most preferably at least 95%, 96%, 97%, 98% or 99% identity with the full length amino acid sequences illustrated herein.

In our co-pending application GB1102091.4 [unpublished] we disclose antigenic polypeptides in vaccines that are protective against bacterial animal pathogens in particular bacterial pathogens of agriculturally important animal species and companion animals and including zoonotic Gram negative bacterial species. The disclosure in GB1102091.4 relates to a class of protective antigen that induces the production of opsonins that target animal [i.e. non-human] bacterial pathogens, for example the cattle/sheep pathogen Mannheimia haemolytica and Haemophilus somnus.

In an alternative preferred embodiment of the invention said polypeptide is isolated from a Gram negative non-human bacterial pathogen.

In a preferred embodiment of the invention said polypeptide is isolated from a Gram negative zoonotic bacterial animal pathogen.

In a preferred embodiment of the invention said non-human bacterial animal pathogen is selected from the genus group consisting of: Mannheimia spp, Actinobacillus spp, Pasteurella spp, Haemophilus spp or Edwardsiella spp.

Additional bacterial pathogens include zoonotic species selected from Brucella spp, Campylobacter spp, Vibrio spp, Yersina spp and Salmonella spp

In a preferred embodiment of the invention said polypeptide is selected from the group
i) an amino acid sequence selected from the group consisting of:SEQ ID NO: 27-42 .
ii) an amino acid sequence as defined in i) above and which is modified by addition, deletion or substitution of one or more amino acid residues and which retains or has enhanced haem binding activity and/or reduced haemolytic activity.

In an alternative preferred embodiment of the invention said polypeptide is selected from the group consisting of:
i) an amino acid sequence selected from the group consisting of: SEQ ID NO:SEQ ID NO: 43, 44, 45, 46, 47, 48, 51, 52, 53, 54, 55 or 56;
ii) an amino acid sequence as defined in i) above and which is modified by addition, deletion or substitution of one or more amino acid residues.

According to an aspect of the invention there is provided a method for immunizing a human against a pathogenic bacterial species comprising:
i) administering an effective amount of a dose of a vaccine composition according to the invention to a human subject to induce protective immunity; optionally
ii) administering one or more further dosages of vaccine to said subject sufficient to induce protective immunity.

According to a further aspect of the invention there is provided a vaccine composition according to the invention for use in the treatment of a bacterial pathogenic infection in a human subject.

According to a further aspect of the invention there is provided a method for the production of an opsonin to an antigen isolated from a human bacterial pathogen comprising:
i) providing a vaccine composition according to the invention;
ii) administering an effective amount of said composition to a human subject sufficient to induce opsonin production.

In an alternative preferred embodiment of the invention said antigenic polypeptide is isolated from a viral pathogen.

In a preferred embodiment of the invention said antigenic polypeptide is derived from a virus selected from the group consisting of: Human Immunodeficiency Virus; Human T Cell Leukaemia Virus; human papilloma virus; papovavirus; rhinovirus; poliovirus; herpesvirus; adenovirus; Epstein Barr virus; influenza virus, hepatitis B and C viruses.

In a preferred embodiment of the invention said antigenic polypeptide comprises an amino acid sequence selected from the group consisting of: SEQ ID NO: 49, 50, 60 or 61.

In a preferred embodiment of the invention said antigenic polypeptide is encoded by a papilloma virus gene, preferably a human papilloma virus gene.

Human papillomaviruses (HPV) vary in their pathological effects. For example, in humans so called low risk HPVs such as HPV-6 and HPV-11 cause benign hyperplasias such as genital warts, (also referred to as condyloma acuminata) while high risk HPVs, for example, HPV-16, HPV-18, HPV-31, HPV-33, HPV-52, HPV-54 and HPV-56, can cause cancers such as cervical or penile carcinoma. HPV-1 causes verruca vulgaris. HPV-5 and HPV-8 cause malignant squamous cell carcinomas of the skin. HPV-2 is found in malignant and non malignant lesions in cutaneous (skin) and squamous (oral) epithelium. HPV-16 is found associated with recurrent respiratory papillomatosis.

In a preferred embodiment of the invention said human papilloma virus is selected from the group consisting of: HPV-2; HPV-6; HPV-11; HPV-16, HPV-18, HPV-31, HPV-33, HPV-52, HPV-54; HPV-56; HPV-5 and HPV-8.

In a preferred embodiment of the invention said HPV is HPV-16.

In a preferred embodiment of the invention said viral gene is E6 or E7.

In a preferred embodiment of the invention said viral gene encodes a polypeptide comprising an amino acid sequence as shown in SEQ ID NO: 62.

In a preferred embodiment of the invention said viral gene encodes a polypeptide comprising an amino acid sequence as shown in SEQ ID NO: 63.

In a still further alternative preferred embodiment of the invention said antigenic polypeptide is an isolated cancer antigen.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Further examples include lung cancer for example small cell lung carcinoma or a non-small cell lung cancer. Other classes of lung cancer include neuroendocrine cancer, sarcoma and metastatic cancers of different tissue origin.

In a preferred embodiment of the invention said cancer antigen is encoded by an oncogene.

In a preferred embodiment of the invention said cancer antigen derived from a cancer gene selected from the group: Her 2, EpCAM or CD19.

In a preferred embodiment of the invention said cancer antigen comprises an amino acid sequence as represented in SEQ ID NO: 58.

In a preferred embodiment of the invention said cancer antigen comprises an amino acid sequence as represented in SEQ ID NO: 59.

In a preferred embodiment of the invention said vaccine composition comprises at least one further adjuvant and/or carrier.

In a preferred embodiment of the invention said further adjuvant is selected from the group consisting of aluminium hydroxide, aluminium or calcium phosphate.

In a preferred embodiment of the invention said further adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative embodiment of the invention said further adjuvant is a TLR agonist such as CpG oligonucleotides, flagellin, monophosphoryl lipid A, poly I:C and derivatives thereof.

In a preferred embodiment of the invention said further adjuvant is a bacterial cell wall derivative such as muramyl dipeptide (MDP) and/or trehalose dicorynomycolate (TDM).

The vaccine compositions of the invention can be administered by any conventional route, including injection, intranasal spray by inhalation of for example an aerosol or nasal drops. The administration may be, for example, intravenous, intraperitoneal, intramuscular, intracavity, subcutaneous, or intradermally. The vaccine compositions of the invention are administered in effective amounts. An “effective amount” is that amount of a vaccine composition that alone or together with further doses, produces the desired response. In the case of treating a particular bacterial disease the desired response is providing protection when challenged by an infective agent.

The amounts of vaccine will depend, of course, on the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used sufficient to provoke immunity; that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of vaccine administered to a subject can be chosen in accordance with different parameters, in particular in accordance with the mode of administration used and the state of the subject. In the event that a response in a subject is insufficient at the initial doses applied, higher doses (or effectively higher doses by a different, more localized delivery route) may be employed to the extent that patient tolerance permits.

In general, doses of vaccine are formulated and administered in effective immunizing doses according to any standard procedure in the art. Other protocols for the administration of the vaccine compositions will be known to one of ordinary skill in the art, in which the dose amount, schedule of injections, sites of injections, mode of administration and the like vary from the foregoing. Administration of the vaccine compositions to mammals other than humans, (e.g. for testing purposes or veterinary therapeutic purposes), is carried out under substantially the same conditions as described above.

In a preferred embodiment of the invention there is provided a vaccine composition according to the invention that includes at least one additional anti-bacterial agent.

In an alternative preferred embodiment of the invention there is provided a vaccine composition according to the invention that includes at least one additional ant-viral agent.

In an alternative preferred embodiment of the invention there is provided a vaccine composition according to the invention that includes at least one additional anti-cancer agent.

Throughout the description and claims of this specification, the words “comprise” and “contain” and variations of the words, for example “comprising” and “comprises”, means “including but not limited to”, and is not intended to and does not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

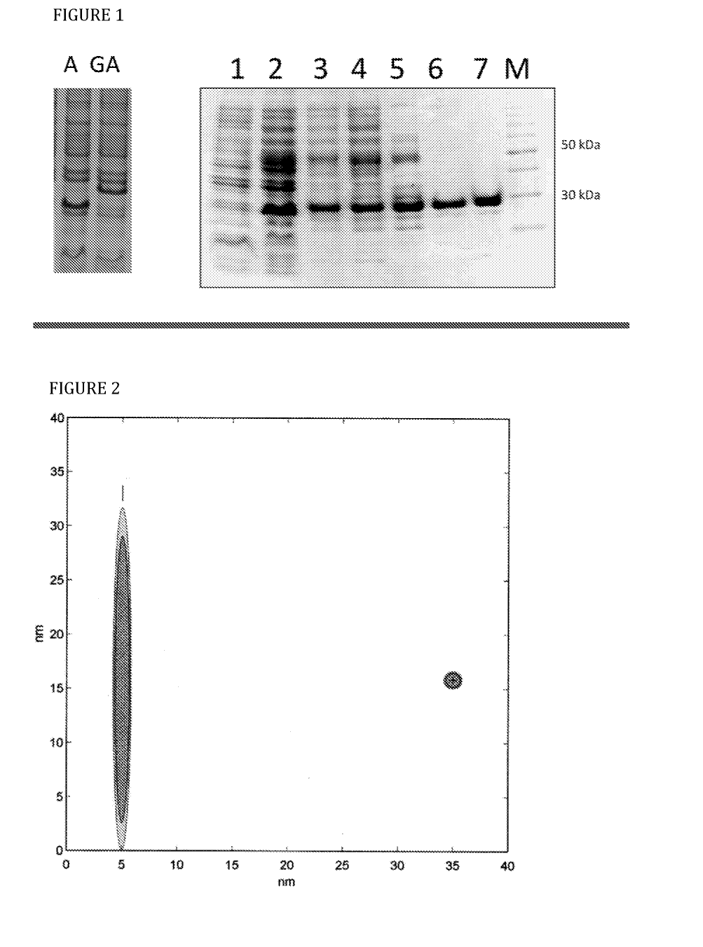

FIG. 1 illustrates expression of α-protein and γ-α-protein and purification of α-protein: SDS-PAGE stained with Coomassie blue showing; Left Panel: Total cell lysates of M72(γ) cells expressing either α-protein (Lane A), or γ-α-protein (lane GA). Right Panel: Lane 1, uninduced cell pellet lysed with SDS; Lane 2, induced cell pellet; Lane 3, soluble protein after treatment with PEI supernatant; Lane 4, proteins from lane 3 after ammonium sulphate precipitation, resuspension and dialysis as applied to Q column; Lane 5, Q column flow-through; Lanes 6 & 7, fractions containing α-protein eluted from SP column; Lane 8, 10 kDa protein size marker

FIG. 2 illustrates sedimentation velocity analysis of α-protein: Shows graphical result of analysis of analytical ultracentrifugation of α-protein, prolate model, Teller method, hydrated a/b=20.026; 2a=31.633 nm; 2b=1.580 nm; hydration expansion=19.88. The model graphs the calculated shape for a protein of 18.8 kDa and the sedimentation coefficient of 1.073 S observed under the conditions of the experiment. Speed: 50 000 rpm. Temp: 22 degrees C. Detection: Rayleigh interference and A₂₄₄. Hydration estimate is 0.552 g water per g protein, based on amino acid composition and solution pH. The calculated frictional ratio (f/f₀) is 2.563; the calculated f/f₀ for a sphere is 1.0.

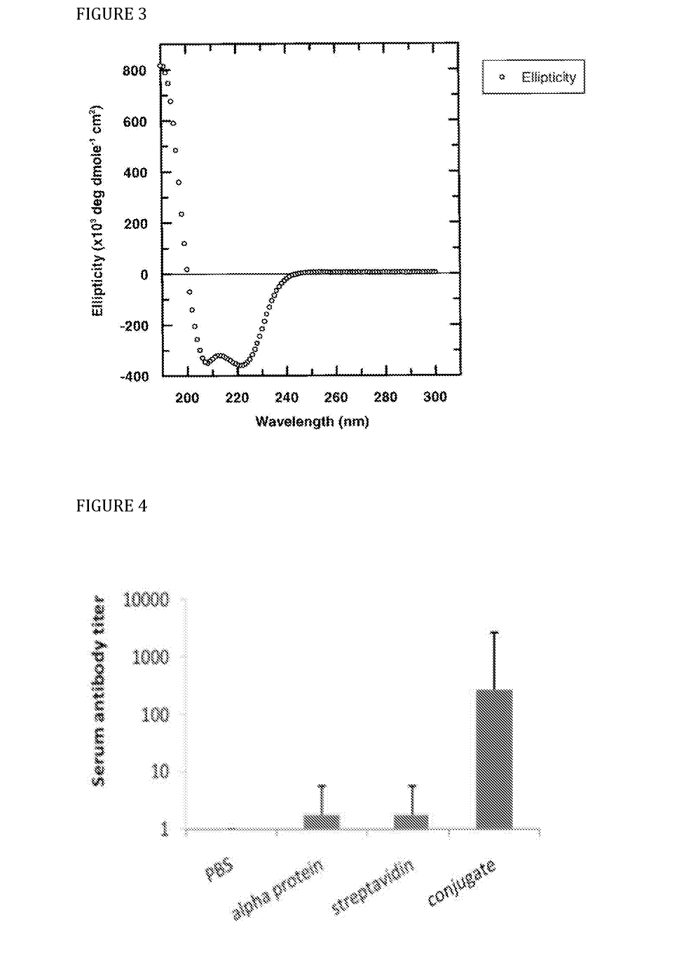

FIG. 3 illustrates circular dichroism of α-protein. The observed curve is characteristic of an all-alpha helical structure.

FIG. 4 illustrates serum antibody titer at Day 13 against streptavidin and alpha protein after one immunization Strepavidin coated 96 well plates were used to determine the presence of mouse anti-streptavidin immunoglobulins in sera from mice inoculated with PBS, α-protein or streptavidin or α-protein-streptavidin complex. Serum antibody titers of the conjugate group against streptavidin in the α-protein-streptavidin-conjugate were higher than that in the streptavidin or control groups.

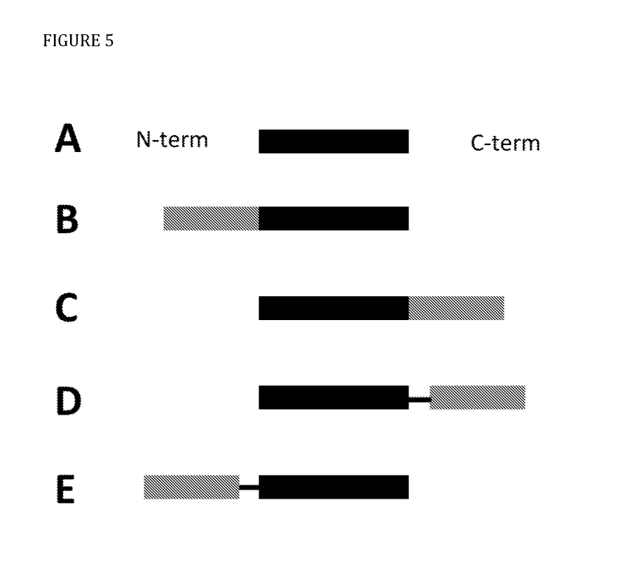

FIG. 5 is a schematic diagram showing possible construction of α-protein fusions: (A) The amino and carboxy termini are indicated on the diagram. The grey boxes represent the proteins of interest, antigen or immunogen and α-proteins are shown as black rectangles. (B) and (C) show fusion at the N or C termini respectively. In (D) and (E) the proteins are linked by a short flexible linker composed of e.g. GGGS or multiple copies thereof.

### MATERIALS AND METHODS

Cloning and Expression of α-protein

DNA encoding the α-protein region of N. meningitidis IgA protease (residues 1003-1171 of the NMB IgA1 protease complete open reading frame accession number AAK15023) was PCR amplified using standard techniques with the following primers Alphaf1: 5′-AAATGAATTCATCGAGGATTTAATTATGAGCCCGCAGGCAAATCAA-3′ incorporating a ribosome binding site and start codon (underlined) and an EcoRI site (italics) and the reverse primer

r1: 5′-ATGACAGAAGCTTTGGTATCTACCTGCGGTTACGACGTTT-3′ which incorporates a HindIII recognition site (italics) and a stop codon (underlined). The PCR products was cloned into the expression vector pJONEX4 (described in Sayers & Eckstein F (1991) Nucleic Acids Res 19: 4127-4132) and transformed into E. coli M72(λ). A fully sequenced individual clone was identified and designated pJONEX_α encoding the α-protein.

Cloning and Expression of γ-α-protein

The region of N. meningitidis IgA protease (residues 977-1171 of the NMB IgA1 protease AAK15023) was PCR amplified using standard techniques with the following primers,

Forward primer F1:

5′-AAATGAATTCATCGAGGATTTAATTATGAGTCCTGCCACAAACACGGC-3′ together with reverse primer R1:

5′-ATGACAGAAGCTTTGGTATCTACCTGCGGTTACGACGTTT-3′ which incorporates a HindIII recognition site (italics) and a stop codon (underlined). PCR product was cloned into the expression vector pJONEX4 (described in Sayers & Eckstein F (1991) Nucleic Acids Res 19: 4127-4132) and transformed into E. coli M72(λ). A fully sequenced individual clone from was identified and designated pJONEX_γ-α encoding γ-α-protein.

Purification of α-protein

Protein was expressed from the pHPNMBa plasmid in E. coli M72(λ) cells and soluble proteins were obtained and subjected to ion exchange chromatography using standard methods. Briefly, the crude protein extract was applied to an anion exchange column (HiTrap Q, Amersham Pharmacia) in 20 mM Tris.HCl pH 8, 1 mM EDTA, 10% glycerol (v/v). The α-protein emerged with the void and was dialysed into 20 mM K₂HPO₄/KH₂PO₄ pH 6.5, 1 mM EDTA, 10% glycerol (v/v) and applied sequentially to HiTrap heparin and SP cation exchange columns (Amersham Pharmacia) and eluted with a linear gradient of NaCl in the same buffer (0-1 M NaCl in 100 ml). Fractions of 5 ml were collected and analysed by SDS-PAGE. Purified protein was stored at −80° C. in 50% glycerol.

Purification of γ-α-protein

Protein was expressed from the pJONEX_γ-α plasmid in E. coli M72(λ) cells and soluble proteins were obtained and subjected to ion exchange chromatography using standard methods. Briefly, the crude protein extract was applied to a cation exchange column (HiTrap heparin, Amersham Pharmacia) in 20 mM K₂HPO₄/KH₂PO₄ pH 5.5, 1 mM EDTA, 1 mM DTT, 5% glycerol (v/v) and eluted with a linear gradient of NaCl in the same buffer (0-1 M NaCl in 100 ml). This was dialysed into 20 mM Tris.HCl pH 8, 1 mM EDTA, 1 mM DTT, 5% glycerol and applied to a HiTrap Q anion exchange column (Amersham Pharmacia) after which protein emerged with the void and was dialysed into 20 mM K₂HPO₄/KH₂PO₄ pH 5.5, 1 mM EDTA, 1 mM DTT 5% glycerol (v/v) and then applied to a cation exchange column (HiTrap heparin, Amersham Pharmacia) in 20 mM K₂HPO₄/KH₂PO₄ pH 5.5, 1 mM EDTA, 1 mM DTT 5% glycerol (v/v). This was eluted with a linear gradient of NaCl in the same buffer (0-1 M NaCl in 100 ml).Fractions of 5 ml were collected and analysed by SDS-PAGE. Purified protein was stored at −20° C. in 50% glycerol.

Synthetic γ Peptide

Synthetic γ peptide to be used for mouse immunisation was ordered from Cambridge Peptides Ltd and was based upon the sequence of the NMB γ peptide cleaved from the α peptide during protein export. The amino acid sequence for the synthetic peptide was as follows; SPATNTASQAQTDSAQIAKPQNIVVAPP [SEQ ID NO: 64].

Immunizations and Antisera Generation

Six- to eight-week-old female BALB/c mice were injected intraperitoneally with 200 μl of phosphate buffered saline containing either 10 μg of α-protein alone, synthetic γ-protein alone, γ-α-protein alone, α-protein and streptavidin, α-protein and synthetic γ-protein, streptavidin alone or streptavidin-α-protein-biotin conjugate. Blood samples were obtained from the tail vein 13 days post inoculation. Blood was kept at 4° C. overnight, and centrifuged for 5 minutes at 13,000×g. The α-protein and/or streptavidin were diluted with PBS to10 μg/ml, and coated 100 μl/well onto ELISA plates (CoStar®). Plates were kept at the 4° C. overnight. The wells were aspirated and washed with wash buffer (PBS+0.05% TWEEN 20). Wells were blocked by adding 100 μl of blocking buffer (PBS+5% milk powder), and incubated at room temperature for 1 hour. Serial dilutions of mouse sera were then added to the wells. After the incubation, the wells were washed with wash buffer, and 100 μl of 1 in 2000 diluted anti-mouse IgG (BD Pharmingen™) linked with horseradish peroxidase (HRP) was added to each well, followed by 1-hour incubation at room temperature. Wells were aspirated and were washed with wash buffer. Then, substrate (SIGMAFAST™ OPD Tablet) for the detection of peroxidase activity in enzyme immunoassays was dissolved in 20 ml water and 100 μl was added to each well. The absorbance was measured at 450 nm wavelength using a Tecan plate-reader with GenS software.

The α-protein and γ-α-protein were readily expressed and the α-protein was readily purified by standard chromatography procedures as shown in FIG. 1. Analytical ultracentrifugation results suggested that it had a very elongated structure (>30 nm in length, FIG. 2). The protein had the expected alpha-helical structure as determined by circular dichroism (FIG. 3).

α-protein Stimulates Immune Response to a Model Antigen

The model antigen (streptavidin) was found to invoke a weak immune response compared to that seen when it was conjugated to α-protein as demonstrated in FIG. 4.

Cross-Linking to α-protein

Purified α-protein was activated with N-hydroxysuccinimide-biotin ester (NHS-biotin) in a potassium phosphate buffer at a pH of 7-8. NHS biotin ester was dissolved in DMSO at 1 mg/ml just before using, and 75 μl (1 mg/ml) was added to 1 ml of protein (1 mg/ml). The reaction was incubated for 4 hours at room temperature, and dialysed against 0.1 M Tris-Cl at pH 8 overnight. Streptavidin (Sigma) was dissolved in phosphate buffered saline (PBS) at 1 mg/ml and then 185 μl of streptavidin was reacted with 500 μl of biotinylated α-protein, and incubated on ice for 1 hour.

Cross-Linking to α-protein General Protocols

Methods for crosslinking proteins and other molecules for immunization are well known in the literature and many may be suitable for linking α-protein to molecules for immunization and vaccination. For example, peptides and proteins containing free cysteine residues can be conjugated to α-protein using heterobifunctional amine-to-thiol crosslinkers such as sulfosuccinimidyl-4-(N-maleimidomethyl)cyclohexane-1-carboxylate (Sulfo-SMCC) under neural pH conditions. The α-protein is first reacted with Sulfo-SMCC under standard conditions and subsequently mixed with the thiol-containing protein or peptide which reacts with the maleimide group in the modified α-protein. Peptides and protein antigens lacking a free thiol can be linked to α-protein using the free amino groups present at the amino terminus or the gamma amino groups of the lysine side chains using the convenient water soluble bis(sulfosuccinimidyl)suberate (Sulfo-DSS), an amine-to-amine crosslinker.

General Construction of α-protein Fusions

The α-protein fusions can be produced using standard recombinant DNA technology including total gene synthesis, as either amino or carboxy terminal-linked α-protein fusions with proteins of interests. The junction between the α-protein and partner may or may not contain a short flexible amino acid linker, also encoded within the recombinant gene. See FIG. 5.

A protein consisting of residues 977-1171 of NMB IgA1 protease, a fusion of the γ-α-protein.

SEQ ID No: 43
MSPATNTASQAQTDSAQIAKPQNIVVAPPSPQANQAEEAKRQQAKAEQVK
RQQAEAERKSAELAKQKAEAEREARELATRQKAEQERSSAELARRHEKER
EAAELSAKQKVEAEREAQALAVRRKAEAEEAKRQAAELARQQEEARKAAE
LAAKQKAETERKAAEIAEQKAEAEREAAELAKQKAEEEGRQAAQSQ

A fusion of the Gly1 protein (accession no: NP_{—}273818) with α-protein at the C-terminus.

SEQ ID No: 44
MKKMFLSAVLLLSAAAQTVWADTVFSCKTDNNKYIEVQKINRNLYEYSFG
SAAKKEIAIRNSKADLLGRSDRWQGMGSGRWATMKFQNGEFMYTIWTGFD
SVTHTESSGVVVERRGKEVARVGCTPKTAQANFNDDDFSSPQANQAEEAK
RQQAKAEQVKRQQAEAERKSAELAKQKAEAEREARELATRQKAEQERSSA
ELARRHEKEREAAELSAKQKVEAEREAQALAVRRKAEAEEAKRQAAELAR
QQEEARKAAELAAKQKAETERKAAEIAEQKAEAEREAAELAKQKAEEEGR
QAAQSQ

Or the mature region of NP_{—}273818 fused to the C-terminus of α protein

SEQ ID No: 45
MSPQANQAEEAKRQQAKAEQVKRQQAEAERKSAELAKQKAEAEREARELA
TRQKAEQERSSAELARRHEKEREAAELSAKQKVEAEREAQALAVRRKAEA
EEAKRQAAELARQQEEARKAAELAAKQKAETERKAAEIAEQKAEAEREAA
ELAKQKAEEEGRQAAQSQAQTVWADTVFSCKTDNNKYIEVQKINRN YEYS
FGSAAKKEIAIRNSKADLLGRSDRWQGMGSGRWATMKFQNGEFMYTIWTG
FDSVTHTESSGVVVERRGKEVARVGCTPKTAQANFNDDDFS

The fusions could contain a small flexible linker region such as GGGS or GGGSGGGS

### EXAMPLE

A fusion of a neisserial Gly1 protein (accession no: NP_{—}273818) with a flexible linker (GGGS) and α-protein at the C-terminus.

SEQ ID No: 46
MKKMFLSAVLLLSAAAQTVWADTVFSCKTDNNKYIEVQKINRNLYEYSFG
SAAKKEIAIRNSKADLLGRSDRWQGMGSGRWATMKFQNGEFMYTIWTGFD
SVTHTESSGVVVERRGKEVARVGCTPKTAQANFNDDDFSGGGSSPQANQA
EEAKRQQAKAEQVKRQQAEAERKSAELAKQKAEAEREARELATRQKAEQE
RSSAELARRHEKEREAAELSAKQKVEAEREAQALAVRRKAEAEEAKRQAA
ELARQQEEARKAAELAAKQKAETERKAAEIAEQKAEAEREAAELAKQKAE
EEGRQAAQSQ

### Example

A fusion of a Mannheimia protein accession no: NP_{—}273818 with the N-terminus of α-protein.

SEQ ID No: 47
MRKLLVITALTLCTTPVFAADKNVIFSCTSTEGKPLTVKRVGNDYEYSYD
KTTFKNPIKKAVTNDGSIIARGSGFTTYALELENDGLKYLVGFVQPNGNA
KEFIEPGATISQRKEQPSIGSVDCDTRKKSHYKFDVHLMNTLSPQANQAE
EAKRQQAKAEQVKRQQAEAERKSAELAKQKAEAEREARELATRQKAEQER
SSAELARRHEKEREAAELSAKQKVEAEREAQALAVRRKAEAEEAKRQAAE
LARQQEEARKAAELAAKQKAETERKAAEIAEQKAEAEREAAELAKQKAEE
EGRQAAQSQ

The N-terminal signal peptide sequence shown in bold (MRKLLVITALTLCTTPVFA) would be removed during expression in E. coli.

### Example

A fusion of a Haemophilus influenzae protein accession no: E1X8Z7 with the N-terminus of α-protein.

SEQ ID No: 48
MKKLLTIGAVAMFATPAFAANNIFSCTAENGSPVSVTKNGSDYEFTYGQV
SFKNPVKQVFANQDSYVATGSGFITSSLEMRNNGTSYTIQFVQPHNSNSI
EEPMLYITNGSKMDTVSCKAGSATQNFERRSMKASSPQANQAEEAKRQQA
KAEQVKRQQAEAERKSAELAKQKAEAEREARELATRQKAEQERSSAELAR
RHEKEREAAELSAKQKVEAEREAQALAVRRKAEAEEAKRQAAELARQQEE
ARKAAELAAKQKAETERKAAEIAEQKAEAEREAAELAKQKAEEEGRQAAQ
SQ

### Example

Hepatitis B virus fused to α-protein C-terminus

SEQ ID No: 49
MSPQANQAEEAKRQQAKAEQVKRQQAEAERKSAELAKQKAEAEREARELA
TRQKAEQERSSAELARRHEKEREAAELSAKQKVEAEREAQALAVRRKAEA
EEAKRQAAELARQQEEARKAAELAAKQKAETERKAAEIAEQKAEAEREAA
ELAKQKAEEEGRQAAQSQENITSGFLGPLLVLQAGFFLLTRILTIPQSLD
SWWTSLNFLGGTTVCLGQNSQSPTSNHSPTSCPPTCPGYRWMCLRRFIIF
LFILLLCLIFLLVLLDYQGMLPVCPLIPGSSTTSAGPCRTCTTTAQGTSM
YPSCCCTKPSDGNCTCIPIPSSWAFGKFLWEWASARFSWLSLLVPFVQWF
AGLSPTVWLSVIWMMWYWGPSLYRILS
PFLPLLPIFFCLWVYI

### Example

|large surface antigen [Hepatitis B virus] fused to α-protein C-terminus

SEQ ID No: 50
MSPQANQAEEAKRQQAKAEQVKRQQAEAERKSAELAKQKAEAEREARELA
TRQKAEQERSSAELARRHEKEREAAELSAKQKVEAEREAQALAVRRKAEA
EEAKRQAAELARQQEEARKAAELAAKQKAETERKAAEIAEQKAEAEREAA
ELAKQKAEEEGRQAAQSQGQNLSTSNPLGFFPDHQLDPAFRANTANPDWD
FNPNKDTWPDANKVGAGAFGLGFTPPHGGLLGWSPQAQGILQTLPANPPP
ASTNRQTGRQPTPLSPPLRNTHPQAMQWNSTTFHQTLQDPRVRGLYFPAG
GSSSGTNPVPTTASPLSSIFSRIGDPALNMENITSGFLGPLLVLQAGFFL
LTRILTIPQSLDSWWTSLNFLGGTTVCLGQNSQSPTSNHSPTSCPPTCPG
YRWMCLRRFIIFLFILLLCLIFLLVLLDYQGMLPVCPLIPGSSTTSTGPC
RTCMTTAQGTSMYPSCCCTKPSDGNCTCIPIPSSWAFGKFLWEWASARFS
WLSLLVPFVQWFVGLSPTVWLSVIVVMMVVYWGPSLYSILSPFLPLLPIF
FCLWVYI

Secreted fibronectin-binding protein antigen fbpA [Mycobacterium tuberculosis accession no.ZP_{—}06445281 fused to α-protein N-terminus

SEQ ID No: 51
MREARMQLVDRVRGAVTGMSRRLVVGAVGAALVSGLVGAVGGTATAGAFS
RPGLPVEYLQVPSPSMGRDIKVQFQSGGANSPALYLLDGLRAQDDFSGWD
INTPAFEWYDQSGLSVVMPVGGQSSFYSDWYQPACGKAGCQTYKWETFLT
SELPGWLQANRHVKPTGSAVVGLSMAASSALTLAIYHPQQFVYAGAMSGL
LDPSQAMGPTLIGLAMGDAGGYKASDMWGPKEDPAWQRNDPLLNVGKLIA
NNTRVWVYCGNGKPSDLGGNNLPAKFLEGFVRTSNIKFQDAYNAGGGHNG
VFDFPDSGTHSWEYWGAQLNAMKPDLQRALGATPNTGPAPQGASPQANQA
EEAKRQQAKAEQVKRQQAEAERKSAELAKQKAEAEREARELATRQKAEQE
RSSAELARRHEKEREAAELSAKQKVEAEREAQALAVRRKAEAEEAKRQAA
ELARQQEEARKAAELAAKQKAETERKAAEIAEQKAEAEREAAELAKQKAE
EEGRQAAQSQ

Based on GNA1870 protein from Neisseria meningitidis fused to the C-terminus of α-protein

SEQ ID No: 52
SPQANQAEEAKRQQAKAEQVKRQQAEAERKSAELAKQKAEAEREARELAT
RQKAEQERSSAELARRHEKEREAAELSAKQKVEAEREAQALAVRRKAEAE
EAKRQAAELARQQEEARKAAELAAKQKAETERKAAEIAEQKAEAEREAAE
LAKQKAEEEGRQAAQSQSSGGGGVAADIGAGLADALTAPLDHKDKSLQSL
TLDQSVRKNEKLKLAAQGAEKTYGNGDSLNTGKLKNDKVSRFDFIRQIEV
DGQLITLESGEFQIYKQDHSAVVALQIEKINNPDKIDSLINQRSFLVSGL
GGEHTAFNQLPDGKAEYHGKAFSSDDAGGKLTYTIDFAAKQGHGKIEHLK
TPEQNVELAAAELKADEKSHAVILGDTRYGSEEKGTYHLALFGDRAQEIA
GSATVKIGEKVHEIGIAGKQ

The following proteins could be used either in chemically cross-linked form with α-protein or constructed as N- or C-terminal fusions by total gene synthesis and expressed in an appropriate host, preferably, E. coli.

Esterase, antigen 85-B [Mycobacterium tuberculosis
CDC1551]
SEQ ID No: 53
MSPLSTLALSAAVTDTEMRRLCARIDIWPPHTVCSGPSTRRHTGQRGTGM
TDVSRKIRAWGRRLMIGTAAAVVLPGLVGLAGGAATAGAFSRPGLPVEYL
QVPSPSMGRDIKVQFQSGGNNSPAVYLLDGLRAQDDYNGWDINTPAFEWY
YQSGLSIVMPVGGQSSFYSDWYSPACGKAGCQTYKWETFLTSELPQWLSA
NRAVKPTGSAAIGLSMAGSSAMILAAYHPQQFIYAGSLSALLDPSQGMGP
SLIGLAMGDAGGYKAADMWGPSSDPAWERNDPTQQIPKLVANNTRLWVYC
GNGTPNELGGANIPAEFLENFVRSSNLKFQDAYNAAGGHNAVFNFPPNGT
HSWEYWGAQLNAMKGDLQSSLGAG
Esterase, [Mycobacterium tuberculosis CDC1551]
SEQ ID No: 54
MHTALHDGGGHMKGRSALLRALWIAALSFGLGGVAVAAEPTAKAAPYENL
MVPSPSMGRDIPVAFLAGGPHAVYLLDAFNAGPDVSNWVTAGNAMNTLAG
KGISVVAPAGGAYSMYTNWEQDGSKQWDTFLSAELPDWLAANRGLAPGGH
AAVGAAQGGYGAMALAAFHPDRFGFAGSMSGFLYPSNTTTNGAIAAGMQQ
FGGVDTNGMWGAPQLGRWKWHDPWVHASLLAQNNTRVWVWSPTNPGASDP
AAMIGQAAEAMGNSRMFYNQYRSVGGHNGHFDFPASGDNGWGSWAPQLGA
MSGDIVGAIR
Chaperonin GroEL [Mycobacterium tuberculosis
H37Rv]
SEQ ID No: 55
MSKLIEYDETARRAMEVGMDKLADTVRVTLGPRGRHVVLAKAFGGPTVTN
DGVTVAREIELEDPFEDLGAQLVKSVATKTNDVAGDGTTTATILAQALIK
GGLRLVAAGVNPIALGVGIGKAADAVSEALLASATPVSGKTGIAQVATVS
SRDEQIGDLVGEAMSKVGHDGVVSVEESSTLGTELEFTEGIGFDKGFLSA
YFVTDFDNQQAVLEDALILLHQDKISSLPDLLPLLEKVAGTGKPLLIVAE
DVEGEALATLVVNAIRKTLKAVAVKGPYFGDRRKAFLEDLAVVTGGQVVN
PDAGMVLREVGLEVLGSARRVVVSKDDTVIVDGGGTAEAVANRAKHLRAE
IDKSDSDWDREKLGERLAKLAGGVAVIKVGAATETALKERKESVEDAVAA
AKAAVEEGIVPGGGASLIHQARKALTELRASLTGDEVLGVDVFSEALAAP
LFWIAANAGLDGSVVVNKVSELPAGHGLNVNTLSYGDLAADGVIDPVKVT
RSAVLNASSVARMVLTTETVVVDKPAKAEDHDHHHGHAH
Co-chaperonin GroES [Mycobacterium tuberculosis
H37Rv]
SEQ ID No: 56
MAKVNIKPLEDKILVQANEAETTTASGLVIPDTAKEKPQEGTVVAVGPGR
WDEDGEKRIPLDVAEGDTVIYSKYGGTEIKYNGEEYLILSARDVLAVVSK
Early secretory antigenic target, 6 kDa
[Mycobacterium tuberculosis CDC1551]
SEQ ID No: 57
MTEQQWNFAGIEAAASAIQGNVTSIHSLLDEGKQSLTKLAAAWGGSGSEA
YQGVQQKWDATATELNNALQNLARTISEAGQAMASTEGNVTGMFA
Human Her2 receptor residues 23-652.
SEQ ID No: 58
TQVCTGTDMKLRLPASPETHLDMLRHLYQGCQVVQGNLELTYLPTNASLS
FLQDIQEVQGYVLIAHNQVRQVPLQRLRIVRGTQLFEDNYALAVLDNGDP
LNNTTPVTGASPGGLRELQLRSLTEILKGGVLIQRNPQLCYQDTILWKDI
FHKNNQLALTLIDTNRSRACHPCSPMCKGSRCWGESSEDCQSLTRTVCAG
GCARCKGPLPTDCCHEQCAAGCTGPKHSDCLACLHFNHSG
Human PSA accession no: P07288 residues 25-261
SEQ ID No: 59
IVGGWECEKHSQPWQVLVASRGRAVCGGVLVHPQWVLTAAHCIRNKSVIL
LGRHSLFHPEDTGQVFQVSHSFPHPLYDMSLLKNRFLRPGDDSSHDLMLL
RLSEPAELTDAVKVMDLPTQEPALGTTCYASGWGSIEPEEFLTPKKLQCV
DLHVISNDVCAQVHPQKVTKFMLCAGRWTGGKSTCSGDSGGPLVCNGVLQ
GITSWGSEPCALPERPSLYTKVVHYRKWIKDTIVANP
HIV gp120 residues 33-511
SEQ ID No: 60
KLWVTVYYGVPVWKEATTTLFCASDAKAYDTEVHNVWATHACVPTDPNPQ
EVVLVNVTENFNMWKNDMVEQMHEDIISLWDQSLKPCVKLTPLCVSLKCT
DLKNDTNTNSSSGRMIMEKGEIKNCSFNISTSIRGKVQKEYAFFYKLDII
PIDNDTTSYKLTSCNTSVITQACPKVSFEPIPIHYCAPAGFAILKCNNKT
FNGTGPCTNVSTVQCTHGIRPVVSTQLLLNGSLAEEEVVIRSVNFTDNAK
TIIVQLNTSVEINCTRPNNNTRKRIRIQRGPGRAFVTIGKIGNMRQAHCN
ISRAKWNNTLKQIASKLREQFGNNKTIIFKQSSGGDPEIVTHSFNCGGEF
FYCNSTQLFNSTWFNSTWSTEGSNNTEGSDTITLPCRIKQIINMWQKVGK
AMYAPPISGQIRCSSNITGLLLTRDGGNSNNESEIFRPGGGDMRDNWRSE
LYKYKVVKIEPLGVAPTKAKRRVVQREKR
HIV GAG protein
SEQ ID No: 61
MGARASVLSGGELDKWEKIRLRPGGKKKYKLKHIVWASRELERFAVNPGL
LETSEGCRQILGQLQPSLQTGSEELRSLYNTVATLYCVHQRIDVKDTKEA
LEKIEEEQNKSKKKAQQAAAAAGTGNSSQVSQNYPIVQNLQGQMVHQAIS
PRTLNAWVKVVEEKAFSPEVIPMFSALSEGATPQDLNTMLNTVGGHQAAM
QMLKETINEEAAEWDRVHPVHAGPIAPGQMREPRGSDIAGTTSTLQEQIG
WMTNNPPIPVGEIYKRWIILGLNKIVRMYSPTSILDIRQGPKEPFRDYVD
RFYKTLRAEQASQDVKNWMTETLLVQNANPDCKTILKALGPAATLEEMMT
ACQGVGGPGHKARVLAEAMSQVTNPANIMMQRGNFRNQRKTVKCFNCGKE
GHIAKNCRAPRKKGCWRCGREGHQMKDCTERQANFLGKIWPSYKGRPGNF
LQSRPEPTAPPEESFRFGEEKTTPSQKQEPIDKELYPLTSLRSLFGNDPS
SQ
E6 [Human papillomavirus type 16]
SEQ ID No: 62
MHQKRTAMFQDPQERPRKLPQLCTELQTTIHDIILECVYCKQQLLRREVY
DFAFRDLCIVYRDGNPYAVCDKCLKFYSKISEYRHYCYSVYGTTLEQQYN
KPLCDLLIRCINCQKPLCPEEKQRHLDKKQRFHNIRGRWTGRCMSCCRSS
RTRRETQL
E7 [Human papillomavirus type 16]
SEQ ID No: 63
MHGDTPTLHEYMLDLQPETTDLYCYEQLNDSSEEEDEIDGPAGQAEPDRA
HYNIVTFCCKCDSTLRLCVQSTHVDIRTLEDLLMGTLGIVCPICSQKP
N. meningitidis γ peptide fusion protein
SEQ ID NO: 64
SPATNTASQAQTDSAQIAKPQNIVVAPP

### Example 1

Serum antibody titre against α- and γ-peptide

Mice were immunized with either α- and γ-peptide, or α- and γ-peptide combined or in fused form and antisera generated. As shown in Table 1 and 2, the α-protein is a highly immunogenic protein when injected alone (Table 1), suggesting that the α-peptide is capable of acting as a carrier protein which increases immune responses to other less immunogenic proteins. The γ-peptide alone failed to illicit an immune response but when injected with the α-peptide in combination or fused form a detectable immune response was measured (Table 2).

Table 1. Serum antibody titres for each group of mice against the α peptide. Serum antibody titres were calculated as the largest dilution factor in which serum antibody titre was greater the base line value derived from mice injected with PBS alone. Mice injected with α alone have a high number of anti-α antibodies. The mice injected with the α+γ mixture have a titre of around 20% less when compared to the α alone group alone. The α-γ fusion shows a 100-fold decrease in antibody titre, whereas γ-alone failed to produce any detectable level of antibodies against the α-peptide.

| Mice immunised with | Serum antibody titre |
|---|---|
| α alone | 12500 |
| γ alone | 0 |
| α + γ mixture | 10000 |
| γ − α fusion | 100 |

Table 2. Serum antibody titres for each group of mice against the γ peptide. Serum antibody titres were calculated as the largest dilution factor in which serum antibody titre was greater the base line value derived from mice injected with PBS alone. As shown mice injected with the α+γ mixture or with the γ-α fusion produced both high levels of antibodies against the γ-protein, whereas the remaining groups failed to produce any detectable levels of anti-γ antibodies.

| Mice immunised with | Serum antibody titre |
|---|---|
| α alone | 0 |
| γ alone | 0 |
| α + γ mixture | 7000 |
| γ − α fusion | 6000 |

1. A composition comprising an adjuvant polypeptide wherein said adjuvant polypeptide comprises a bacterial α-protein cross-linked or associated with an antigenic molecule to which an immune response is desired.

2. The composition according to claim 1, wherein said α-protein comprises a polypeptide that includes at least one amino acid motif comprising amino acid residues EAERXAAELAXX(K/Q) (SEQ ID NO: 69) wherein X is any hydrophilic amino acid residue.

3. The composition according to claim 1, wherein said antigenic molecule is a polypeptide antigen.

4. The composition according to claim 1, wherein said antigenic molecule is a polysaccharide antigen.

5. The composition according to claim 1, wherein said antigenic molecule is a lipopolysaccharide antigen.

6. The composition according to claim 3, wherein the α-protein and the polypeptide antigen are in frame translational fusion proteins.

7. The composition according to claim 6, wherein said polypeptide antigen is a bacterial pathogen.

8. The composition according to claim 7 wherein said polypeptide antigen is selected from the group consisting of:
i) an amino acid sequence selected from the group consisting of SEQ ID NOs: 1-26; and
ii) an amino acid sequence as defined in i) above and which is modified by addition, deletion or substitution of one or more amino acid residues and which retains or has enhanced haemolytic activity and/or reduced haemolytic activity.

9. The composition according to claim 7, wherein said polypeptide antigen is selected from the group consisting of:
i) an amino acid sequence selected from the group consisting of SEQ ID NOs: 27-42; and
ii) an amino acid sequence as defined in i) above and which is modified by addition, deletion or substitution of one or more amino acid residues and which retains or has enhanced haemolytic activity and/or reduced haemolytic activity.

10. The composition according to claim 7, wherein said polypeptide antigen is selected from the group consisting of:
i) an amino acid sequence selected from the group consisting of SEQ ID NO: 43, 44, 45, 46, 47, 48, 51, 52, 53, 54, 55 and 56; and
ii) an amino acid sequence as defined in i) above and which is modified by addition, deletion or substitution of one or more amino acid residues.

11. The composition according to claim 7, wherein said polypeptide antigen comprises or consists of the amino acid sequence in SEQ ID: NO 64 or an amino acid sequence which is modified by addition, deletion or substitution of one or more amino acid residues.

12. A method for stimulating an immune response a against a pathogenic bacterial species comprising:
i) administering an effective amount of a dose of the composition of claim 7 to a human subject to induce protective immunity; and optionally
ii) administering one or more further dosages of the composition to said subject sufficient to induce protective immunity.

13. (canceled)

14. A method for producing an opsonin to an antigen isolated from a human bacterial pathogen, comprising:
i) providing the composition according to claim 8; and
ii) administering an effective amount of said composition to a human subject sufficient to induce opsonin production.

15. The composition according to claim 3, wherein said polypeptide antigen is a viral pathogen.

16. The composition according to claim 15, wherein said polypeptide antigen comprises an amino acid sequence selected from the group consisting of: SEQ ID NOs: 49, 50, 60 and 61.

17. The composition according claim 15, wherein said polypeptide antigen is encoded by a papilloma virus gene.

18. The composition according claim 17, wherein said viral gene is E6 or E7.

19. The composition according to claim 18, wherein said viral gene encodes a polypeptide comprising the amino acid sequence shown in SEQ ID NO: 62.

20. The composition according to claim 18, wherein said viral gene encodes a polypeptide comprising the amino acid sequence shown in SEQ ID NO: 63.

21. The composition according to claim 3, wherein said polypeptide antigen is cancer antigen.

22. The composition according to claim 21, wherein said cancer antigen is encoded by an oncogene.

23. The composition according to claim 21, wherein said cancer antigen is derived from a cancer gene selected from the group consisting of Her 2, EpCAM and CD19.

24. The composition according to claim 21 wherein said cancer antigen comprises the amino acid sequence shown in SEQ ID NO: 58.

25. The composition according to claim 21, wherein said cancer antigen comprises the amino acid sequence shown in SEQ ID NO: 59.

26. The composition according to claim 1, wherein said composition comprises at least one additional adjuvant and/or a carrier.


SEQUENCE LISTING (71 sequences)

SEQ ID NO: 1 - protein, 139 aa, Neisseria meningitidis
MKKMFLSAVLLLSAAAQTVWADTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFS

SEQ ID NO: 2 - protein, 118 aa, Neisseria meningitidis
DTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFS

SEQ ID NO: 3 - protein, 140 aa, Neisseria gonorrhoeae
MKKMFLSAALLLSAAAQTVWADTVFSCKTDNNKYIEVQKINRNLYEYSFGSAAKKEIAIRNSKADLLGRSDRWQGMGSGRRATMKFQNGEFMYTVWTGFDSVTHTESSGVVVERRGKEVARVGCTPKTAQANFNDDDFSR

SEQ ID NO: 4 - protein, 119 aa, Neisseria gonorrhoeae
DTVFSCKTDNNKYIEVQKINRNLYEYSFGSAAKKEIAIRNSKADLLGRSDRWQGMGSGRRATMKFQNGEFMYTVWTGFDSVTHTESSGVVVERRGKEVARVGCTPKTAQANFNDDDFSR

SEQ ID NO: 5 - protein, 140 aa, Neisseria cinerea
MKRTLILIFSGMIGTAGIAQAGSVFSCKTDNNKYIEVQKINRNLYEYSFGSAAKKEIAIRNSKADLLGRSDRWQGIGRGRWATMKFQNGEFMYTVWTDFDSVTHTESSGVVVERRGKEVARVGCTPKTAQANFDDDDFSW

SEQ ID NO: 6 - protein, 119 aa, Neisseria cinerea
GSVFSCKTDNNKYIEVQKINRNLYEYSFGSAAKKEIAIRNSKADLLGRSDRWQGIGRGRWATMKFQNGEFMYTVWTDFDSVTHTESSGVVVERRGKEVARVGCTPKTAQANFDDDDFSW

SEQ ID NO: 7 - protein, 152 aa, Providencia alcalifaciens
MKLRRTSIAIVLPILMSAPVLADSHTVTVFQCVNKNDRQVKVTLINGQYQYQFGKLNQQPDIEITRSPKQLTQGFFQTRYADTDTGYAASQEIDFRNGAYTYTIFTNHSGSKIESGVEVYNKDKRLTKVVCVPNTVVDNIYEHLSEIPERPE

SEQ ID NO: 8 - protein, 130 aa, Providencia alcalifaciens
DSHTVTVFQCVNKNDRQVKVTLINGQYQYQFGKLNQQPDIEITRSPKQLTQGFFQTRYADTDTGYAASQEIDFRNGAYTYTIFTNHSGSKIESGVEVYNKDKRLTKVVCVPNTVVDNIYEHLSEIPERPE

SEQ ID NO: 9 - protein, 149 aa, Proteus penneri
MKARIAIAAVFVLLSASVYAEEFTCQLENGKYVSVFVEHGKPPVYRYGTLAKTEITLPVPQQPNNNVFYGHQMFVAGASTYIRFKNGNYSYIVYDGEGRGWNFNGIIVYKDNNIISKKECKQPLTPSLDKLKNYSIKMDPYLETYIYAP

SEQ ID NO: 10 - protein, 129 aa, Proteus penneri
EEFTCQLENGKYVSVFVEHGKPPVYRYGTLAKTEITLPVPQQPNNNVFYGHQMFVAGASTYIRFKNGNYSYIVYDGEGRGWNFNGIIVYKDNNIISKKECKQPLTPSLDKLKNYSIKMDPYLETYIYAP

SEQ ID NO: 11 - protein, 153 aa, Providencia rustigianii
MYKKIAVCLALGLFTASASAVEYTCQLENNKTVSVVVETGKVPVYRYGTLGKTEITLPINAKGQESIYVGQGTFSAGGSIYIRFQNGPFSYLLYDGEGRGWYFQGIAVYKNGKLVNKKSCKPDSNLNLYSLLDYGIPTDPDEDGPYTFDPNSN

SEQ ID NO: 12 - protein, 133 aa, Providencia rustigianii
VEYTCQLENNKTVSVVVETGKVPVYRYGTLGKTEITLPINAKGQESIYVGQGTFSAGGSIYIRFQNGPFSYLLYDGEGRGWYFQGIAVYKNGKLVNKKSCKPDSNLNLYSLLDYGIPTDPDEDGPYTFDPNSN

SEQ ID NO: 13 - protein, 157 aa, Providencia stuartii
MNKTLLGMVIIGVSATPVMAAEFTCQLENKKYVSVIVDPGKTPQYRYGTLAKPEITLPLNEKGARNIFVGQGHFIAGESLYIRFQNGPYSYLLYQGEGRGWAFKGLAVYKNNKLINKKECQNWNSFDLYDILNYQIRTDPALELYDSPYSFDPNSNE

SEQ ID NO: 14 - protein, 137 aa, Providencia stuartii
AEFTCQLENKKYVSVIVDPGKTPQYRYGTLAKPEITLPLNEKGARNIFVGQGHFIAGESLYIRFQNGPYSYLLYQGEGRGWAFKGLAVYKNNKLINKKECQNWNSFDLYDILNYQIRTDPALELYDSPYSFDPNSNE

SEQ ID NO: 15 - protein, 138 aa, Vibrio cholerae
MSIFTLFSSILLMSLHVNAQTIFYCETQNNKKVEVQIVGDLLQYRFGEKLYKPEIEVLVARRKTSTFQWLGVGMDEYYDVTIPNRDVFYKVFTSRERKPEGKLEAGISVWSKDKLIAEIYCNNDSLYEVLFDIELPKE

SEQ ID NO: 16 - protein, 119 aa, Vibrio cholerae
QTIFYCETQNNKKVEVQIVGDLLQYRFGEKLYKPEIEVLVARRKTSTFQWLGVGMDEYYDVTIPNRDVFYKVFTSRERKPEGKLEAGISVWSKDKLIAEIYCNNDSLYEVLFDIELPKE

SEQ ID NO: 17 - protein, 140 aa, Pseudomonas mendocina
MMRIFFVLVFSICFPLVVNGSESHMPIFMCKTKNNKNIEVYRDGDMVTYSFGRSDYPPELKLTRSMADLEVAIGSVSGNEISNSITFSNGVYAYRVMTTINKVSATQEPRHGVLVKKKTKYLAYIDCVPETVQGSLLDLE

SEQ ID NO: 18 - protein, 120 aa, Pseudomonas mendocina
SESHMPIFMCKTKNNKNIEVYRDGDMVTYSFGRSDYPPELKLTRSMADLEVAIGSVSGNEISNSITFSNGVYAYRVMTTINKVSATQEPRHGVLVKKKTKYLAYIDCVPETVQGSLLDLE

SEQ ID NO: 19 - protein, 135 aa, Haemophilus influenzae
MKKLLTIGAVAMFATPAFAANNIFSCTAENGSPVSVTKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKAS

SEQ ID NO: 20 - protein, 116 aa, Haemophilus influenzae
ANNIFSCTAENGSPVSVTKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKAS

SEQ ID NO: 21 - protein, 135 aa, Haemophilus parainfluenzae
MKKLLTIAAVAVCATPAFAANNIFSCTAENGSPVSVVKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKVS

SEQ ID NO: 22 - protein, 116 aa, Haemophilus parainfluenzae
ANNIFSCTAENGSPVSVVKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKVS

SEQ ID NO: 23 - protein, 176 aa, Cronobacter turicensis
MVALWGLLREEMLTNYVKRFFYEIYIFYFFIMFFYNCHAAGCNVEENLVASCDLPGQIKRTAAFCADKNNKIKYYFKESHAIKFKIEFNSERKLKRWLDLGTYTTYFGFNNASYTYVLGVPEEKPGALAFLEIKKNGKTISSKECSSNSFGEKNIESDSIEDVPDSVVRGDFFKFP

SEQ ID NO: 24 - protein, 137 aa, Cronobacter turicensis
AGCNVEENLVASCDLPGQIKRTAAFCADKNNKIKYYFKESHAIKFKIEFNSERKLKRWLDLGTYTTYFGFNNASYTYVLGVPEEKPGALAFLEIKKNGKTISSKECSSNSFGEKNIESDSIEDVPDSVVRGDFFKFP

SEQ ID NO: 25 - protein, 173 aa, Burkholderia spp
MPRDRIAELIVALSMLNVAHAEGLCRSSETTVFNCDLKENHKIVSICSSKDLTDKSGFLQYRYGSRNKVEFAFPKSLTNSQSQFGYDEYSRPDLSTFVVGFNYENYRYEISETTEGSDDDALTTRTLLVSSGTTTRGLKLTCLDNQNTVSDISTLDHVLKCDKKPAIIEGACD

SEQ ID NO: 26 - protein, 152 aa, Burkholderia
EGLCRSSETTVFNCDLKENHKIVSICSSKDLTDKSGFLQYRYGSRNKVEFAFPKSLTNSQSQFGYDEYSRPDLSTFVVGFNYENYRYEISETTEGSDDDALTTRTLLVSSGTTTRGLKLTCLDNQNTVSDISTLDHVLKCDKKPAIIEGACD

SEQ ID NO: 27 - protein, 142 aa, Mannheimia haemolytica
MRKLLVITALTLCTTPVFAADKNVIFSCTSTEGKPLTVKRVGNDYEYSYDKTTFKNPIKKAVTNDGSIIARGSGFTTYALELENDGLKYLVGFVQPNGNAKEFIEPGATISQSKEQPSIGSVDCDTRKKSHYKFDVHLMNTL

SEQ ID NO: 28 - protein, 123 aa, Mannheimia haemolytica
ADKNVIFSCTSTEGKPLTVKRVGNDYEYSYDKTTFKNPIKKAVTNDGSIIARGSGFTTYALELENDGLKYLVGFVQPNGNAKEFIEPGATISQSKEQPSIGSVDCDTRKKSHYKFDVHLMNTL

SEQ ID NO: 29 - protein, 143 aa, Mannheimia haemolytica
MKKMAIVALSAFFSMNAFANNNVVYSCTTTDNQTLKVTKEGGNYVYSHGNTTFKNPVKEALKNPASEIAGGSQFTTISLELRNAGKSYIVGHIEADPKSPFEASVQIQDIKTGNDITSFECRSDKPIRHNFDRKLMRKSGFAA

SEQ ID NO: 30 - protein, 124 aa, Mannheimia haemolytica
NNNVVYSCTTTDNQTLKVTKEGGNYVYSHGNTTFKNPVKEALKNPASEIAGGSQFTTISLELRNAGKSYIVGHIEADPKSPFEASVQIQDIKTGNDITSFECRSDKPIRHNFDRKLMRKSGFAA

SEQ ID NO: 31 - protein, 137 aa, Actinobacillus pleuropneumoniae
MRKLTTLAVCSFISLPVLAENISIFSCTAEDGNQILIQKTGADYQFSYGKILFKNPAKQVLKNEGSYVATGSGFTTSALEMKSKSHSYTIEFVQPRGTNVIDSPMLYITKGNNTTEIKCDTNKKVGQHFEYSKMNIL

SEQ ID NO: 32 - protein, 118 aa, Actinobacillus pleuropneumoniae
ENISIFSCTAEDGNQILIQKTGADYQFSYGKILFKNPAKQVLKNEGSYVATGSGFTTSALEMKSKSHSYTIEFVQPRGTNVIDSPMLYITKGNNTTEIKCDTNKKVGQHFEYSKMNIL

SEQ ID NO: 33 - protein, 154 aa, Pasteurella dagmatis
MKKHILFTNHPKYKECPMKKITLITLLFFVSAATANNVVEDIDYQQEEPIFYCKAPNKKIIQLTKYEEVYIYRFGKNLDKPEIRLENHIEDVKRTSETQNGFETKRAYELTTGPYIYRIFSGIPKVPEENGVEVFKHRKRIARIECSGYVHIFE

SEQ ID NO: 34 - protein, 119 aa, Pasteurella dagmatis
NNVVEDIDYQQEEPIFYCKAPNKKIIQLTKYEEVYIYRFGKNLDKPEIRLENHIEDVKRTSETQNGFETKRAYELTTGPYIYRIFSGIPKVPEENGVEVFKHRKRIARIECSGYVHIFE

SEQ ID NO: 35 - protein, 130 aa, Pasteurella multocida
MKSVYFFLPFVCASFSLNAANTSIPMLDKSYQESSQQQEEPIFTCRTNQGKIIQLNKYDKVYIYRFGANLNNPELTLTSHIENIIKGVITKGAIEIKRFYELTSGNYTYHIFYTNPKAYDENGAEVFKNN

SEQ ID NO: 36 - protein, 111 aa, Pasteurella multocida
ANTSIPMLDKSYQESSQQQEEPIFTCRTNQGKIIQLNKYDKVYIYRFGANLNNPELTLTSHIENIIKGVITKGAIEIKRFYELTSGNYTYHIFYTNPKAYDENGAEVFKNN

SEQ ID NO: 37 - protein, 142 aa, Haemophilus somnus
MRKLLGITVLTLCTTSAFATDKNVVFSCTSTEGKPLTVKRVGNDYEYSYDKTIFKNPIKKAVTNDGSIIARGSGFTTYALELKNDGLKYVVGFVQPNGNSKEFIEPGATISRDNDQPSIGSVDCDPYKKIYYKFDVHLMNTL

SEQ ID NO: 38 - protein, 123 aa, Haemophilus somnus
TDKNVVFSCTSTEGKPLTVKRVGNDYEYSYDKTIFKNPIKKAVTNDGSIIARGSGFTTYALELKNDGLKYVVGFVQPNGNSKEFIEPGATISRDNDQPSIGSVDCDPYKKIYYKFDVHLMNTL

SEQ ID NO: 39 - protein, 164 aa, Edwardsiella ictaluri
MGRVIRIGRYGGLVSLPISGVQTAADPLLFQCGLDNGMVARLTLYDGVPRYGYGVMKHGRIYSPPLTLPRQEGERAMVRCGRSVLPGATYCRFINVNYRYVVMDSIGHDRSFTGVRVFHERKLVSPLNCRNGGFVISDYHAQGLIRDTDTGDEGAYGYLLPDAG

SEQ ID NO: 40 - protein, 140 aa, Edwardsiella ictaluri
ADPLLFQCGLDNGMVARLTLYDGVPRYGYGVMKHGRIYSPPLTLPRQEGERAMVRCGRSVLPGATYCRFINVNYRYVVMDSIGHDRSFTGVRVFHERKLVSPLNCRNGGFVISDYHAQGLIRDTDTGDEGAYGYLLPDAG

SEQ ID NO: 41 - protein, 145 aa, Haemophilus somnus
MRHKKLLFSTLAMVTFLTIMPVQAATKPIFSCSTAQGKKLLVEKTGSNYVLSYQDLKVTNPISQILERTNSHIVSRSGYVLYSLEFSDPKYSYYMQYQESMGDEKPLFAGLFRVEPNSNNAPTTLVKCNLKKPISNNFEINLMNN

SEQ ID NO: 42 - protein, 121 aa, Haemophilus somnus
ATKPIFSCSTAQGKKLLVEKTGSNYVLSYQDLKVTNPISQILERTNSHIVSRSGYVLYSLEFSDPKYSYYMQYQESMGDEKPLFAGLFRVEPNSNNAPTTLVKCNLKKPISNNFEINLMNN

SEQ ID NO: 43 - protein, 196 aa, Artificial Sequence
  Fusion protein
MSPATNTASQAQTDSAQIAKPQNIVVAPPSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 44 - protein, 306 aa, artificial sequence
  Fusion protein
MKKMFLSAVLLLSAAAQTVWADTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFSSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 45 - protein, 292 aa, artificial sequence
  Fusion protein
MSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQAQTVWADTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFS

SEQ ID NO: 46 - protein, 310 aa, artificial sequence
  Fusion protein
MKKMFLSAVLLLSAAAQTVWADTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFSGGGSSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 47 - protein, 309 aa, artificial sequence
  Fusion protein
MRKLLVITALTLCTTPVFAADKNVIFSCTSTEGKPLTVKRVGNDYEYSYDKTTFKNPIKKAVTNDGSIIARGSGFTTYALELENDGLKYLVGFVQPNGNAKEFIEPGATISQRKEQPSIGSVDCDTRKKSHYKFDVHLMNTLSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 48 - protein, 302 aa, artificial seqoence
  Fusion protein
MKKLLTIGAVAMFATPAFAANNIFSCTAENGSPVSVTKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKASSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 49 - protein, 393 aa, Artificial Sequence
  Fusion protein
MSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSAGPCRTCTTTAQGTSMYPSCCCTKPSDGNCTCIPIPSSWAFGKFLWEWASARFSWLSLLVPFVQWFAGLSPTVWLSVIWMMWYWGPSLYRILSPFLPLLPIFFCLWVYI

SEQ ID NO: 50 - protein, 555 aa, Artificial sequence
  Fusion protein
MSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAMQWNSTTFHQTLQDPRVRGLYFPAGGSSSGTNPVPTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 51 - protein, 510 aa, Artificial sequence
  Fusion protein
MREARMQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGASPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQ

SEQ ID NO: 52 - protein, 420 aa, Artificial sequence
  Fusion protein
SPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARQQEEARKAAELAAKQKAETERKAAEIAEQKAEAEREAAELAKQKAEEEGRQAAQSQSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 53 - protein, 374 aa, Mycobacterium tuberculosis
MSPLSTLALSAAVTDTEMRRLCARIDIWPPHTVCSGPSTRRHTGQRGTGMTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 54 - protein, 310 aa, Mycobacterium tuberculosis
MHTALHDGGGHMKGRSALLRALWIAALSFGLGGVAVAAEPTAKAAPYENLMVPSPSMGRDIPVAFLAGGPHAVYLLDAFNAGPDVSNWVTAGNAMNTLAGKGISVVAPAGGAYSMYTNWEQDGSKQWDTFLSAELPDWLAANRGLAPGGHAAVGAAQGGYGAMALAAFHPDRFGFAGSMSGFLYPSNTTTNGAIAAGMQQFGGVDTNGMWGAPQLGRWKWHDPWVHASLLAQNNTRVWVWSPTNPGASDPAAMIGQAAEAMGNSRMFYNQYRSVGGHNGHFDFPASGDNGWGSWAPQLGAMSGDIVGAIR

SEQ ID NO: 55 - protein, 539 aa, Mycobacterium tuberculosis
MSKLIEYDETARRAMEVGMDKLADTVRVTLGPRGRHVVLAKAFGGPTVTNDGVTVAREIELEDPFEDLGAQLVKSVATKTNDVAGDGTTTATILAQALIKGGLRLVAAGVNPIALGVGIGKAADAVSEALLASATPVSGKTGIAQVATVSSRDEQIGDLVGEAMSKVGHDGVVSVEESSTLGTELEFTEGIGFDKGFLSAYFVTDFDNQQAVLEDALILLHQDKISSLPDLLPLLEKVAGTGKPLLIVAEDVEGEALATLVVNAIRKTLKAVAVKGPYFGDRRKAFLEDLAVVTGGQVVNPDAGMVLREVGLEVLGSARRVVVSKDDTVIVDGGGTAEAVANRAKHLRAEIDKSDSDWDREKLGERLAKLAGGVAVIKVGAATETALKERKESVEDAVAAAKAAVEEGIVPGGGASLIHQARKALTELRASLTGDEVLGVDVFSEALAAPLFWIAANAGLDGSVVVNKVSELPAGHGLNVNTLSYGDLAADGVIDPVKVTRSAVLNASSVARMVLTTETVVVDKPAKAEDHDHHHGHAH

SEQ ID NO: 56 - protein, 100 aa, Mycobacterium tuberculosis
MAKVNIKPLEDKILVQANEAETTTASGLVIPDTAKEKPQEGTVVAVGPGRWDEDGEKRIPLDVAEGDTVIYSKYGGTEIKYNGEEYLILSARDVLAVVSK

SEQ ID NO: 57 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 58 - protein, 240 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSG

SEQ ID NO: 59 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 60 - protein, 479 aa, Human deficiency virus
KLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 61 - protein, 502 aa, Human deficiency virus
MGARASVLSGGELDKWEKIRLRPGGKKKYKLKHIVWASRELERFAVNPGLLETSEGCRQILGQLQPSLQTGSEELRSLYNTVATLYCVHQRIDVKDTKEALEKIEEEQNKSKKKAQQAAAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQDVKNWMTETLLVQNANPDCKTILKALGPAATLEEMMTACQGVGGPGHKARVLAEAMSQVTNPANIMMQRGNFRNQRKTVKCFNCGKEGHIAKNCRAPRKKGCWRCGREGHQMKDCTERQANFLGKIWPSYKGRPGNFLQSRPEPTAPPEESFRFGEEKTTPSQKQEPIDKELYPLTSLRSLFGNDPSSQ

SEQ ID NO: 62 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 63 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 64 - protein, 28 aa, Artificial Sequence
  Synthetic peptide
SPATNTASQAQTDSAQIAKPQNIVVAPP

SEQ ID NO: 65 - DNA, 46 bp, artificial sequence
  Primer
aaatgaattcatcgaggatttaattatgagcccgcaggcaaatcaa

SEQ ID NO: 66 - DNA, 40 bp, artificial sequence
  Primer
atgacagaagctttggtatctacctgcggttacgacgttt

SEQ ID NO: 67 - DNA, 48 bp, artificial sequence
  Primer
aaatgaattcatcgaggatttaattatgagtcctgccacaaacacggc

SEQ ID NO: 68 - DNA, 40 bp, artificial sequence; identical to SEQ ID NO: 66
  Primer

SEQ ID NO: 69 - protein, 13 aa, Artificial Sequence
  alpha protein
EAERXAAELAXXX

SEQ ID NO: 70 - protein, 4 aa, Artificial Sequence
  linker sequence
GGGS

SEQ ID NO: 71 - protein, 8 aa, Artificial Sequence
  linker sequence
GGGSGGGS